(19) United States
(12) Patent Application Publication
(10) Pub. No.: US 2006/0159723 A1
(43) Pub. Date: Jul. 20, 2006

(54) Antimicrobial lenses and methods of their use

(76) Inventors: David C. Turner, Jacksonville, FL (US); Azaam Alli, Jacksonville, FL (US); James D. Ford, Orange Park, FL (US); Stephen L. Galas, Jacksonville, FL (US); Ann-Marie W. Meyers, Jacksonville, FL (US); Frank L. Neely, Jacksonville, FL (US); James R. Petisce, Jacksonville, FL (US); Robert B. Steffen, Jacksonville Beach, FL (US); Douglas G. Vanderlaan, Jacksonville, FL (US); James Jen, Jacksonville, FL (US); Joseph R. Hepting, Jacksonville, FL (US); James Ebel, Glendale, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/291,462

(22) Filed: Dec. 1, 2005

Related U.S. Application Data
(62) Division of application No. 10/029,526, filed on Dec. 21, 2001.
(60) Provisional application No. 60/309,642, filed on Aug. 2, 2001.

(51) Int. Cl. C09D 5/16; A61K 33/38

(52) U.S. Cl. 424/427; 424/618; 523/122

(57) ABSTRACT

This invention relates to antimicrobial lenses containing coated zeolites and methods for their production.

### RELATED PATENT APPLICATIONS

This patent application claims priority of a provisional application, U.S. Ser. No. 60/309,642 which was filed on Aug. 2, 2001.

### FIELD OF THE INVENTION

This invention relates to antimicrobial lenses as well as methods of their production, and use.

### BACKGROUND OF THE INVENTION

Contact lenses have been used commercially to improve vision since the 1950s. The first contact lenses were made of hard materials. They were used by a patient during waking hours and removed for cleaning. Current developments in the field gave rise to soft contact lenses, which may be worn continuously, for several days or more without removal for cleaning. Although many patients favor these lenses due to their increased comfort, these lenses can cause some adverse reactions to the user. The extended use of the lenses can encourage the buildup of bacteria or other microbes, particularly, Pseudomonas aeruginosa, on the surfaces of soft contact lenses. The build-up of bacteria and other microbes can cause adverse side effects such as contact lens acute red eye and the like. Although the problem of bacteria and other microbes is most often associated with the extended use of soft contact lenses, the build-up of bacteria and other microbes occurs for users of hard contact lens wearers as well.

Therefore, there is a need to produce contact lenses that inhibit the growth of bacteria or other microbes and/or the adhesion of bacterial or other microbes on the surface of contact lenses. Further there is a need to produce contact lenses which do not promote the adhesion and/or growth of bacteria or other microbes on the surface of the contact lenses. Also there is a need to produce contact lenses that inhibit adverse responses related to the growth of bacteria or other microbes.

Others have recognized the need to produce soft contact lenses that inhibit the growth of bacteria or other microbes. One reference discloses that silver, a known antimicrobial agent, can be incorporated into contact lenses using a silver zeolite to give an antimicrobial lens. This reference, EP 1050314 A1, teaches that a certain weight percentage of silver zeolites can be molded into a lens. However, the teaching of this reference does not solve the problem of microbial growth or adhesion on contact lenses.

The antimicrobial effect of the lenses of EP 1,050,314 is caused by the exchange of silver between the zeolites and the surrounding tissues. However, since the zeolites of EP 1,050,314 rapidly release silver, the antimicrobial activity of these lenses reduces rapidly as silver diffuses into the ocular environment and the surrounding tissues. In some cases it has been shown that lenses containing silver zeolites lose their antimicrobial effect in less than 24 hours. For lenses that are meant to be used for a period of a week or more, an antimicrobial effect of less than 24 hours is insufficient. Therefore there exists a need to produce lenses whose antimicrobial effect extends for more than 24 hours. This need is filled by the invention described below.

### BRIEF DESCRIPTION OF THE DRAWINGS

FIG. 1 Lens Movement versus Silver Content

### DETAILED DESCRIPTION OF THE INVENTION

This invention includes an antimicrobial lens comprising, consisting essentially of, or consisting of a coated zeolite.

As used herein, the term, “antimicrobial lens” means a lens that exhibits one or more of the following properties, the inhibition of the adhesion of bacteria or other microbes to the lenses, the inhibition of the growth of bacteria or other microbes on lenses, and the killing of bacteria or other microbes on the surface of lenses or in an area surrounding the lenses. For purposes of this invention, adhesion of bacteria or other microbes to lenses, the growth of bacteria or other microbes on lenses and the presence of bacterial or other microbes on the surface of lenses is collectively referred to as “microbial colonization.” Preferably, the lenses of the invention exhibit at least a 1-log reduction (≧90% inhibition) of viable bacteria or other microbes, more preferably a 2-log reduction (≧99% inhibition) of viable bacteria or other microbes. Such bacteria or other microbes include but are not limited to those organisms found in the eye, particularly Pseudomonas aeruginosa, Acanthamoeba species, Staphyloccus aureus, E. coli, Staphyloccus epidermidis, and Serratia marcesens.

As use herein, the term “zeolites” means an aluminosilicate having a three dimensional skeletal structure that is generally represented by xM_{2/n} O.Al₂O₃.ySiO₂.zH₂O, written with Al₂O₃ as a basis, wherein M represents an ion-exchangeable cationic species, which is usually the ion of a monovalent or divalent metal; n corresponds to the valence of the metal; x is a coefficient of the metal oxide; y is a coefficient of silica; and z is the number of waters of crystallization. The metal-component of the zeolite includes metals that have antimicrobial activity such as silver. copper, zinc, mercury, tin, lead, bismuth, cadmium, chromium, cobalt, nickel or a combination of two or more of these metals. Aside from metals M can be other cationic species for example ammonium cations such as tetramethylammonium. Often zeolites contain a mixture of metals, including metals that do not confer antimicrobial activity. Examples of these metal cations include potassium, sodium, calcium, and the like. These metals may be present in zeolites of the invention in addition to the metals that confer antibacterial activity. The preferred antimicrobial metals are silver, zinc, and copper and the particularly preferred metal is silver.

There are known various kinds of zeolites having different particle diameters, component ratios, and specific surface areas. Any natural or synthetic zeolites can be used in the present invention.

Examples of natural zeolites include analcime, chabazite, clinoptilolite, erionite, faujasite, mordenite, and phillipsite. Examples of synthetic zeolites include A-type zeolite, X-type zeolite, Y-type zeolite, and mordenite. In the present invention synthetic zeolites are the preferred zeolites. The particle diameter of the zeolites can vary, from about 10 to about 5000 nanometer (nm), preferably about 10 to about 400 nm, more preferably, about 10 to about 200 nm, most preferably about 50 to about 160 nm.

The antimicrobial activity of lenses of the invention varies with the amount of antimicrobial metal present in the zeolites. If the antimicrobial metal content of the zeolites is measured before the zeolites are incorporated into the lenses or the lenses are used on a patient, the initial percentage of antibacterial metal in the zeolites is about 1% to about 50%, based on total weight of the zeolite. Preferably the antibacterial metal content of the zeolites is about 8% to about 30%, more preferably about 10% to about 20%.

The preferred zeolites of the invention are syntnetic A-type zeolites or Y-type zeolites with silver ions. The average particle diameter of the zeolites is about 10 to about 1200 nm, preferably about 10 to less than about 200 nm, most preferably about 50 nm to about 100 nm. The initial silver content of the preferred zeolites in the lenses of this invention is about 10% to about 20%. “Coated zeolites” refer to the zeolites that are treated with hydrophobic substances that slow the release of the antimicrobial metal. Substances that are useful to coat zeolites include but are not limited to silanes, hydrophobic monomers, and mixtures thereof. To obtain coated zeolites, the zeolites may be stirred, sprayed, sonicated, or heated, where the preferred method of obtaining coated zeolites is by stirring the zeolites in hydrophobic substances.

The silanes useful in this invention are compounds represented by the following Formula I preferably with a molecular weight of about 600 or less (this is multiplied in the case of the oligomer): 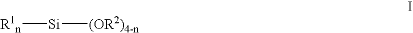
wherein R¹ is a monovalent hydrophobic group such as C₁₋₂₀alkyl, C₁₋₈alkenyl, phenyl, phenylC₁₋₈alkyl, haloC₁₋₈alkyl, fluoroC₁₋₈alkyl, C₁₋₈alkoxycarbonylC₁₋₈alkyl, C₁₋₈alkylsiloxy; R² is C₁₋₆alkyl, C₁₋₈alkenyl phenyl, phenylC₁₋₈alkyl, haloC₁₋₈alkyl, or C₁₋₈alkoxycarbonylC₁₋₈alkyl and n is 1-3. The preferred R¹ is C₁₋₂₀alkyl the particularly preferred R¹ is saturated C₁₈alkyl. The preferred R² is C₁₋₃alkyl the particularly preferred R² is methyl and the preferred n is 3.

In general, silanes of Formula (II) can be used in which R¹ and n are defined as for Formula (I), and in which X is any group that can be displaced by a nucleophile. The preferred X is chloro, bromo, iodo, acyloxy, hydroxyl, or NH—Si(CH₃)₃.

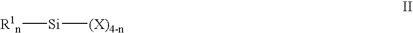

Examples of useful silanes of Formula I and Formula II include but are not limited to phenyltrimethoxysilane, phenyltriethoxysilane, diphenyldimethoxysilane, diphenyidiethoxysilane, methyltrimethoxysilane, methyltriethoxysilane, methyltripropoxysilane, ethyltrimethoxysilane, ethyltriethoxysilane, ethyltripropoxysilane, propyltrimethoxysilane, propyltriethoxysilane, propyltripropoxysilane, butyltrimethoxysilane, butyltriethoxysilane, hexyltrimethoxysilane, hexyltriethoxysilane, benzyltrimethoxysilane, octyltrimethoxysilane, octyltriethoxysilane, octyltripropoxysilane, decyltrimethoxysilane, dodecyltrimethoxysilane, octadecyltrimethoxysilane, tetradecyltrimethoxysilane, tetradecyltriethoxysilane, hexadecyltrimethoxysilane, hexadecyltriethoxysilane, dimethyldimethoxysilane, dimethyldiethoxysilane, dibutyldimethoxysilane, octadecylmethyldimethoxysilane, octadecyidimethylmethoxysilane, acetoxypropyltrimethoxysilane, octadecyltrichlorosilane, trifluoropropyltrimethoxysilane, perfluorodecyl-1H,1H,2H,2H-dimethylchlorosilane, N-(2-aminoethyl)-3-aminopropyltrimethoxysilane, and 3-aminopropyltrimethoxysilane. Alternatively, a condensed dimer or trimer or higher oligomers of the aforesaid silane may be used. The oligomers may be used as long as they are hydrolyzable. Alternatively, other silanes that are capable of reacting with the silanol groups on the surfaces of zeolites can be used. Disilazanes, such as hexamethyldisilazane, can also be used. The preferred silanes of the invention are octadecyltrimethoxy silane, octyltrimethoxysilane, butyltrimethoxysilane, and acetoxypropyltrimethoxysilane, octadecyltrichlorosilane where octadecyltrimethoxysilane is particularly preferred.

To coat the zeolites with silanes, zeolites are stirred with silanes under slightly acidic or alkaline conditions. When alkoxysilanes, such as octadecyltrimethoxy silane are used, the pH of the stirred mixture is adjusted with acetic acid to about 4 to about 5.5. Alternatively, alkoxysilanes, such as octadecyl trimethoxysilane, may be stirred with zeolites and a sufficient amount of a tertiary amine (ex. triethylamine) to adjust the pH to about 10 to about 12. When chlorosilanes, disilazanes, or aminosilanes are used no pH adjustment is required.

The hydrophobic monomers that are useful in this invention include but are not limited to perfluoropropylene oxide, diethylene glycol vinyl ether, methyl methacrylate, lauryl methacrylate, styrene, 1,3-butadiene, propylene glycol, hexamethylcyclotrisiloxane, and mixtures thereof. These hydrophobic monomers can be coated to the surface of the zeolites using the plasma treatment methods described in V. Panchalingam, X. Chen, C. R. Savage, R. B. Timmons and R. C. Eberhart, J. Appl. Polm. Sci.: Appl. Polym. Symp., 54, 123 (1994) or modifications of that procedure such as replacing the stationary glass plasma chamber with a rotating plasma chamber, or varying the wattage across the electrodes. Alternatively, the monomers can be coated on the surface of the zeolites via free-radical or anionic polymerization methods. The preferred hydrophobic monomers for use with plasma treatment are a mixture of perfluoropropylene oxide and diethylene glycol vinyl ether.

As used herein, the term “lens” refers to an ophthalmic device that resides in or on the eye. These devices can provide optical correction or may be cosmetic. The term lens includes but is not limited to soft contact lenses, hard contact lenses, intraocular lenses, overlay lenses, ocular inserts, and optical inserts. Soft contact lenses are made from silicone elastomers or hydrogels, which include but are not limited to silicone hydrogels, and fluorohydrogels. Preferably, the lenses of the invention are optically clear, with optical clarity comparable to currently available commercial lenses such as lenses made from etafilicon A, genfilcon A, lenefilcon A, polymacon, acquafilcon A, balafilcon A, and lotrafilcon A.

Coated zeolites of the invention may be added to the soft contact lens formulations described in U.S. Pat. No. 5,710,302, WO 9421698, EP 406161, JP 2000016905, U.S. Pat. No. 5,998,498, U.S. patent application Ser. No. 09/532,943, U.S. Pat. No. 6,087,415, U.S. Pat. No. 5,760,100, U.S. Pat. No.5,776, 999, U.S. Pat. No. 5,789,461, U.S. Pat. No. 5,849,811, and U.S. Pat. No. 5,965,631. In addition, coated zeolites of the invention may be added to the formulations of commercial soft contact lenses. Examples of commercially available soft contact lenses formulations include but are not limited to the formulations of etafilcon A, genfilcon A, lenefilcon A, polymacon, acquafilcon A, balafilcon A, and lotrafilcon A. The preferable contact lens formulations are etafilcon A, balafilcon A, acquafilcon A, lotrafilcon A, and silicone hydrogels, as prepared in U.S. Pat. No. 5,998,498, U.S. patent application Ser. No. 09/532,943, a continuation-in-part of U.S. patent application Ser. No. 09/532,943, filed on Aug. 30, 2000, U.S. Pat. No. 6,087,415, U.S. Pat. No. 5,760,100, U.S. Pat. No. 5,776, 999, U.S. Pat. No. 5,789,461, U.S. Pat. No. 5,849,811, and U.S. Pat. No. 5,965,631. These patents as well as all other patent disclosed in this paragraph are hereby incorporated by reference in their entirety. The amount of coated zeolites contained in the lenses of the invention is about 0.01% to about 20%, preferably, about 0.02% to about 1.0%, more preferably, about 0.025% to about 0.3%. When silver zeolites are used in the invention, the silver content of the lenses of the invention ranges from about 0.001 wt % (weight percent) to about 5 wt %.

Hard contact lenses are made from polymers that include but are not limited to polymers of poly(methyl)methacrylate, silicon acrylates, fluoroacrylates, fluoroethers, polyacetylenes, and polyimides, where the preparation of representative examples may be found in JP 200010055, JP 6123860 and U.S. Pat. No. 4,330,383. Intraocular lenses of the invention can be formed using known materials. For example, the lenses may be made from a rigid material including, without limitation, polymethyl methacrylate, polystyrene, polycarbonate, or the like, and combinations thereof. Additionally, flexible materials may be used including, without limitation, hydrogels, silicone materials, acrylic materials, fluorocarbon materials and the like, or combinations thereof. Typical intraocular lenses are described in WO 0026698, WO 0022460, WO 9929750, WO 9927978, WO 0022459, and JP 2000107277. U.S. Pat. Nos. 4,301,012; 4,872,876; 4,863,464; 4,725,277; 4,731,079. Coated zeolites may be added to hard contact lens formulations and intraocular lens formulations in the same manner and at the same percentage as described above for soft contact lenses. All of the references mentioned in this application are hereby incorporated by reference in their entirety.

Lenses prepared from coated zeolites and the aforementioned formulations may be coated with a number of agents that are used to coat lens. This additional external lens coating may be used to increase the comfort of the lenses or to further slow down the release of silver to the surrounding tissues. For example, the coating procedures, compositions, and methods of U.S. Pat. Nos. 6,087,415, 5,779,943, 5,275,838, 4,973,493, 5,135,297, 6,193,369, 6,213,604, 6,200,626, and 5,760,100 may be used and these patents are hereby incorporated by reference for those procedures, compositions, and methods.

Further, the invention includes an antimicrobial lens comprising, consisting of, or consisting essentially of, a coated zeolite having a duration of antimicrobial activity greater than that of a lens comprising a non-coated zeolite.

The terms lens, antimicrobial, coated zeolite, and zeolite all have their aforementioned meanings and preferred ranges. The phrase “duration of antimicrobial activity” means the amount of time that the lenses of the invention reduce microbial colonization on the lenses. The duration of antimicrobial activity can be tested by a broth assay or a vortex assay.

In the vortex assay a culture of Pseudomonas aeruginosa, ATCC# 15442 (ATCC, Rockville, Md.) was grown overnight in a nutrient medium. The bacterial inoculum was prepared to result in a final concentration of approximately 1×108 colony forming units/mL. Three contact lenses were rinsed with phosphate buffered saline (PBS) pH 7.4±0.2. Each rinsed contact lens was combined with two (2) mL of bacterial inoculum into a sterile glass vial, which was rotated in a shaker-incubator (100 rpm) for two (2) hrs. at 37±2° C. Each lens was rinsed with PBS to remove loosely bound cells, placed into 10 mL of PBS containing 0.05% w/v Tween™ 80 and vortexed at 2000 rpm for three minutes. The resulting supernatant was enumerated for viable bacteria, and the results, reported of the detected viable bacteria attached to three lenses were averaged.

In the biological broth assay, lenses of the invention are washed with Dulbecco's Phosphate Buffered Saline without calcium chloride and magnesium chloride, are placed into 1000 μl of Mueller Hinton Broth containing approxiamtely 10⁸ cfu/ml Pseudomonas aeruginosa (ATCC 15442), and are incubated at 37±2° C. overnight. The resulting solutions were observed for opacity and cultured to enumerate the bacteria, and compared to similar lenses without coated zeolites.

Although the lenses of the invention may not sustain the same level of activity for the duration of its recommended use, the lenses of the invention sustain their antimicrobial activity for a longer period of time than lenses prepared from uncoated zeolites.

Still further, the invention includes a method of reducing the adverse effects associated with microbial colonization in the ocular regions of a mammal comprising, consisting of, or consisting essentially of, placing an antimicrobial lens comprising a coated zeolite on the eye of a mammal.

The terms lens, antimicrobial lens, and coated zeolite all have their aforementioned meanings and preferred ranges. The phrase “adverse effects associated with microbial colonization” include but are not limited to contact ocular inflammation, contact lens related peripheral ulcers, contact lens associated red eye, infiltrative keratitis, microbial keratitis, and the like. The term mammal means any warm blooded higher vertebrate, and the preferred mammal is a human.

Yet further, the invention includes a method of producing an antimicrobial lens comprising, consisting essentially of, or consisting of a coated zeolite

where the method comprises, consists essentially of, or consists of the steps of
(a) coating a zeolite with a silane or with a hydrophobic monomer to produce a coated zeolite
(b) adding the coated zeolite of step (a) to a lens formulation prior to curing said lens formulation.
   The terms lens, antimicrobial lens, and hydrophobic monomer, coated zeolite all have their aforementioned meanings and preferred ranges. The coating of the zeolites can be accomplished by a number of methods which include but are not limited to stirring, spraying, sonicating, plasma coating, or heating the zeolite with a silane or a hydrophobic monomer.

Yet further still, the invention includes a method of coating a zeolite with a silane comprising contacting the zeolite with the silane at a pH of about greater than 4 and about less than 5.5.

Yet even further still, the invention includes a method of coating a zeolite with a silane comprising contacting the zeolite with a silane at a pH of about greater than 10 and about less than 12.

Even, yet further still, the invention includes a method of producing an antimicrobial lens comprising, consisting essentially of, or consisting of a coated zeolite

where the method comprises, consists essentially of, or consists of the steps of
(a) coating a zeolite containing a non-antimicrobial metal with a silane or a hydrophobic monomer to form a coated zeolite;
(b) adding the zeolite of step (a) to a lens formulation prior to curing said lens formulation;
(c) curing the lens formulation to produce a lens and
(d) treating the lens of step (d) with an solution containing soluble salts of an antimicrobial metal.
   The terms lens, antimicrobial lens, and coated zeolite all have their aforementioned meanings and preferred ranges. The term “non-antimicrobial metal” refers to metals that impart little or no antimicrobial activity to zeolites and lenses made from those zeolites. The non-antimicrobial metals include but are not limited to potassium, sodium, and calcium. The preferred non-antimicrobial metal is sodium. The antimicrobial metals are metals that confer antimicrobial activity to the zeolites and lenses made from those zeolites. The preferred antimicrobial metals are silver, copper, and zinc, or a combination thereof. If the antimicrobial metal is silver, the soluble salts of that metal include but are not limited to silver nitrate, silver acetate, silver citrate, silver sulfate, and silver picrate. Said soluble salts may be present in a concentration of about 0.5% to about 20%, (weight/weight; w/w), preferably about 5%. The preferred solutions are aqueous solutions.

Providing a lens that fits a wide range of patients has been a quest of eye care practitioners and lens manufacturers for a number of years. In order to produce such a lens, many variables, such as lens material, design, surface treatments, and additional components such as ophthalmic drugs, tints, dyes and pigments can come into play. For example it has been shown that if one adds too much of an additional component, such as an antimicrobial agent, a lens that will become adhered to the eye is produced. However, if one is attempting to produce an antimicrobial lens, a balance should be struck between producing a lens that contains enough antimicrobial agent to produce the desired effect without producing a lens that adheres to the eye.

One way to assess if a lens fit is acceptable (i.e. the lens is not adhered) is to assess the tightness of the fit of a lens. (Young, G. et al., Influence of Soft Contact Lens Design on Clinical Performance, Optometry and Vision Science, Vol 70, No., 5 pp. 394-403) Tightness of a lens may be assessed using an in vivo push up test. In that test, a lens is placed on a patient's eye. Subsequently, an eye care practitioner presses his or her finger digitally upward against the lower lid of the patient's eye and observes whether the lens moves on the patient's eye (Id.). Lenses that do not move under these circumstances are not considered to be a good fit for the patient's eye, for lenses that are too tight will not move when the patient blinks and may become uncomfortable. Therefore one of the objects of this invention is to produce an antimicrobial lens that does not adhere to the patient's eye.

To meet this objective, the invention includes an antimicrobial lens comprising, consisting essentially of, or consisting of silver, wherein said lens has sufficient movement on the eye of a patient, provided that the lens does not contain significant amounts of un-coated zeolites having a diameter of greater that 200 nm.

The terms lens, antimicrobial lens, all have their aforementioned meanings and preferred ranges. The phrase “movement on the eye of a patient” refers to whether a lens, when placed on the eye of a patient moves under the push-up test described above. This test is described in further detail in Contact Lens Practice, Chapman & Hall, 1994, edited by M. Ruben and M. Guillon, pgs. 589-99. Under this test lenses are given an −2 rating if they do not move on the eye of a patient in the digital push-up test. Therefore lenses that score greater than a “−2” on the digital push-up test are lenses that move on a patient's eye. In a statistically significant patient population, lenses that may be suitable for one patient may not be suitable for another. Therefore, lenses having sufficient movement are lenses that move on at least about 50 to about 100% of a given patient population. Preferably, said lenses move on about 75 to about 100%, of patients, more preferably, about 80 to about 100%, most preferably about 90 to about 100%.

The term “silver” refers to silver metal of any oxidation state (Ag⁰, Ag¹⁺ or Ag²⁺) that Is incorporated into a lens, where the preferred oxidation state is oxidized silver. The amount of silver that is incorporated into the lenses ranges from about 20 ppm to about 100,000 ppm, where any lens containing at least about 20 ppm has antimicrobial properties. The preferred amount of silver that is incorporated into the lens is about 20 ppm to about 4,000 ppm, more preferably, 20 ppm to about 1,500 ppm, even more preferably about 30 ppm to about 600 ppm.

Lenses containing zeolites or coated zeolites are one way of producing an antibacterial lens that contains silver and have sufficient movement on the eye of a patient. However, they are not only lenses containing silver that may have sufficient movement. Other methods of incorporating into contact lenses may be used, provided that those methods produce lenses having sufficient movement on the eye of a patient. For example, lenses containing monomers that reversibly bind to silver (“Monomers of 030”) are another way of producing such a lens. The preparation and use of lenses containing Monomers of 030 is disclosed in U.S. Provisional App. Ser. No. 60/257,030, filed on Dec. 21, 2000 and in a U.S. patent application entitled “Antimicrobial Contact Lenses And Methods For Their Production,” that was filed on Dec. 20, 2001 and claims priority from the provisional application. This reference is hereby incorporated by reference in its entirety. In addition to the methods disclosed in the filed application, one can bind silver to Monomers of 030 prior to incorporation into lens formulations to produce lenses containing silver and Monomers of 030.

Another method of incorporating silver into lenses is to treat a lens that does not contain silver with a silver containing solution. Therefore, the invention includes a method of adding silver to an antimicrobial lens, comprising, consisting essentially of, or consisting of heating a lens with a silver containing solution.

Silver may be added to the lens by washing the cured and hydrated lens in a silver solution such as silver nitrate in deionized water (“DI”). Other sources of silver include but are not limited to silver acetate, silver citrate, silver iodide, silver lactate, silver picrate, and silver sulfate. The concentration of silver in these solutions can vary from the concentration required to add a known quantity of silver to a lens to a saturated silver solution. In order to calculate the concentration of the silver solution needed, the following calculation is used: the concentration of silver solution is equal to the desired amount of silver per lens, multiplied by the dry weight of the lens divided by the total volume of treating solution.
silver solution concentration (μg/mL)=[desired silver in lens (μg/g)×average dry lens weight (g)]/total volume of treating solution (mL)
   For example, if one requires a lens containing 40 μg/of silver, the dry weight of the lens is 0.02. g, and the vessel used to treat said lens has a volume of 3 mL, the required silver concentration would be 0.27 μg/mL.

As used herein “heating” has its common meaning where the temperature at which the lens is heated is from about 40 to about 130° C.

Yet another method of incorporating silver into lenses is to add silver salts to lens formulations. Silver salts that may be added include but are not limited to silver acetate, silver citrate, silver iodide, silver lactate, silver picrate, and silver sulfate.

Yet, still another method of incorporating silver into lenses is to produce lenses containing nano-sized zeolites. Therefore the invention includes an antimicrobial lens comprising, consisting essentially of, or consisting of nano-sized zeolites.

The terms, lens, antimicrobial lens, silver, and zeolites all have their aforementioned meanings and preferred ranges. The term “nano-sized” refers to the diameter of the zeolites. The diameter of the nano-sized zeolites used in this invention is about 10 to about 200 nanometers (nm). preferably, about 10 to about 150 nm, most preferably about 50 nm to about 100 nm.

Still, yet another method of incorporating silver into lenses is to produce lenses containing silver and an oxidizing agent. Often when silver is incorporated into lenses, the lenses turn from clear to a discolored appearance over time. This discoloration may compromise the visual acuity of the lens and can be esthetically unappealing to the patient. Therefore, preventing or reducing discoloration is a goal of any lens producer. To meet this goal, the invention includes an antimicrobial lens comprising silver and an oxidizing agent.

The terms, antimicrobial, lens, and silver all have their aforementioned meanings and preferred ranges. “Oxidizing agents” are substances that remove an electron from Ag⁰ to produce Ag⁺¹ or Ag⁺². Oxidizing agents include but are not limited to hydrogen peroxide, organic peroxides such as, peracetic acid, performic acid, perbenzoic acid, or inorganic oxidants such as sodium hypochlorite, potassium permanganate, oxygen, iodine, sodium iodate, nitric acid, sodium or potassium nitrate, sodium peroxide, sodium or potassium periodate, sodium or potassium perchlorate, potassium persulfate, sodium perborate, and potassium peroxydiphosphate. The preferred oxidants for use in this invention are those with good water solubility and low toxicity such as hydrogen peroxide, oxygen, sodium or potassium nitrate and sodium hypochlorite. The most preferred oxidant is hydrogen peroxide. Oxidizing agents are added to contact lens formulations prior to curing at a concentration of about 10 to about 1000 ppm.

In addition to preparing antimicrobial lenses containing silver and oxidizing agents, there are other methods of reducing discoloration in lenses containing silver that are prone to discoloration. Therefore, the invention includes a method of reducing discoloration in an antimicrobial lens comprising, consisting essentially of or consisting of contacting said antimicrobial lens with an oxidizing agent.

The terms, antimicrobial, lens, and oxidizing agent all have their aforementioned meanings and preferred ranges. The term “contacting” includes any means of placing the oxidizing agent in close physical proximity with the lens. The most common method of contacting is to prepare an aqueous solution of the oxidizing agent and to stir, soak, or otherwise mix the lens in said solution.

In order to illustrate the invention the following examples are included. These examples do not limit the invention. They are meant only to suggest a method of practicing the invention. Those knowledgeable in contact lenses as well as other specialties may find other methods of practicing the invention. However, those methods are deemed to be within the scope of this invention.

### EXAMPLES

The following abbreviations were used in the examples
BAGE=glycerin esterified with boric acid
Bloc-HEMA=2-(trimethylsiloxy) ethyl methacrylate
Blue HEMA=the reaction product of reactive blue number 4 and HEMA, as described in Example 4 or U.S. Pat. No. 5,944,853
CGI 1850=1:1 (w/w) blend of 1-hydroxycyclohexyl phenyl ketone and bis (2,6-dimethyoxybenzoyl)-2,4-4-trimethylpentyl phosphine oxide
DI water=deionized water
D3O=3,7-dimethyl-3-octanol
EGDMA=ethyleneglycol dimethacrylate
EO₂V=diethylene glycol vinyl ether
DMA N,N-dimethylacrylamide
DAROCUR 1173 2-hydroxy-2-methyl-1-phenyl-propan-1-one
HEMA=hydroxyethyl methacrylate
60% IPA=Isopropyl alcohol, 60% v/v DI
MM=methacrylic acid;
MMA=methyl methacrylate
TMI=dimethyl meta-isopropenyl benzyl isocyanate
mPDMS=mono-methacryloxypropyl terminated polydimethylsiloxane (MW 800-1000)
Norbloc=2-(2′-hydroxy-5-methacrylyloxyethylphenyl)-2H-benzotriazole
PVP=polyvinylpyrrolidinone (K 90)
TM=t-amyl alcohol
TBACB=tetrabutyl ammonium-m-chlorobenzoate
TEGDMA tetraethyleneglycol dimethacrylate
THF=tetrahydrofuran
TRIS=tis(trimethylsiloxy)-3-methacryloxypropylsilane
TMPTMA=trimethylolprbpane trimethacrylate
w.w=weighvtotal weight
w/v=weighttotal volume
v/v=volume/total volume
3M3P=3-methyl-3-pentanol.
   The formulations that were used to prepare the lenses of the invention were prepared as follows.
   Macromer 2 Preparation

To a dry container housed in a dry box under nitrogen at ambient temperature was added 30.0 g (0.277 mol) of bis(dimethylamino)methylsilane, a solution of 13.75 mL of a 1M solution of TBACB (386.0 g TBACB in 1000 mL dry THF), 61.39 g (0.578 mol) of p-xylene, 154.28 g (1.541 mol) methyl methacrylate (1.4 equivalents relative to initiator), 1892.13 (9.352 mol) 2-(trimethylsiloxy)ethyl methacrylate (8.5 equivalents relative to initiator) and 4399.78 9 (61.01 mol) of THF. To a dry, three-necked, round-bottomed flask equipped with a thermocouple and condenser, all connected to a nitrogen source, was charged the above mixture prepared in the dry box.

The reaction mixture was cooled to 15° C. while stirring and purging with nitrogen. After the solution reached 15° C., 191.75 g (1.100 mol) of 1-trimethylsiloxy-1-methoxy-2-methylpropene (1 equivalent) was injected into the reaction vessel. The reaction was allowed to exotherm to approximately 62° C. and then 30 mL of a 0.40 M solution of 154.4 g TBACB in 11 mL of dry THF was metered in throughout the remainder of the reaction. After the temperature of reaction reached 30° C. and the metering began, a solution of 467.56 g (2.311 mol) 2-(trimethylsiloxy)ethyl methacrylate (2.1 equivalents relative to the initiator), 3636.6. 9 (3.463 mol) n-butyl monomethacryloxypropyl-polydimethylsiloxane (3.2 equivalents relative to the initiator), 3673.84 g (8.689 mol), TRIS (7.9 equivalents relative to the initiator) and 20.0 g bis(dimethylamino)methylsilane was added.

The mixture was allowed to exotherm to approximately 38-42° C. and then allowed to cool to 30° C. At that time, a solution of 10.0 g (0.076 mol) bis(dimethylamino)methylsilane, 154.26 g (1.541 mol) methyl methacrylate (1.4 equivalents relative to the initiator) and 1892.13 g (9.352 mol) 2-trimethylsiloxy)ethyl methacrylate (8.5 equivalents relative to the initiator) was added and the mixture again allowed to exotherm to approximately 40° C. The reaction temperature dropped to approximately 30° C. and 2 gallons of THF were added to decrease the viscosity. A solution of 439.69 g water, 740.6 g methanol and 8.8 g (0.068 mol) dichloroacetic acid was added and the mixture refluxed for 4.5 hours to de-block the protecting groups on the HEMA. Volatiles were then removed and toluene added to aid in removal of the water until a vapor temperature of 110° C. was reached.

The reaction flask was maintained at approximately 110° C. and a solution of 443 g (2.201 mol) TMI and 5.7 g (0.010 mol) dibutyltin dilaurate were added. The mixture was reacted until the isocyanate peak was gone by IR. The toluene was evaporated under reduced pressure to yield an off-white, anhydrous, waxy reactive monomer. The macromer was placed into acetone at a weight basis of approximately 2:1 acetone to macromer. After 24 hrs, water was added to precipitate out the macromer and the macromer was filtered and dried using a vacuum oven between 45 and 60° C. for 20-30 hrs.

Macromer 1 Preparation

The procedure for Macromer 2 used except that 19.1 mole parts HEMA, 5.0 mole parts MAA, 2.8 mole parts MMA; 7.9 mole parts TRIS, 3.3, mole parts mPDMS, and 2.0 mole parts TMI were used.

Macromer 3 Preparation

The procedure for Macromer 2 was used except that 19.1 mole parts HEMA, 7.9 mole parts TRIS, 3.3 mole parts mPDMS, and 2.0 mole parts TMI were used.

Marcromer 4 Preparation

The procedure for Macromer 2 was used except that dibutyltin dilaurate was replaced with triethylamine.

### EXAMPLE 1

### Preparation of Octadecyl Trimethoxysilane Coated Zeolites

Type A zeolite particles (15.0 grams, average particle size 1000 nm to 2000 nm) containing 10% silver by weight were added to methanol (150 mL). Glacial acetic acid (9 μL) and octadecytrimethoxysilane (15 mL) were added and the suspension was stirred at room temperature for 24 hours. The solvent was removed by vacuum filtration to give a solid. This solid was re-suspended in ethanol and isolated by vacuum filtration 3 times. The resulting solid was dried under vacuum to give Zeolite A¹ as a fine powder.

### EXAMPLE 2

### Preparation of Lenses A¹

A hydrogel blend was made from the following monomer mix (all amounts were calculated as weight percent of the total weight of the combination): 17.98% Macromer 2, 28.0% mPDMS, 14.0% TRIS, 26.0% DMA, 5.0% HEMA, 1.0% TEGDMA, 5.0% PVP, 1.0% CGI 1850, 2.0% Norbloc, and 0.02% Blue HEMA. To 80 part (wgt) of this blend were added 0.19 parts Zeolites of Example 1, 1.0 part acetic acid (when Macromer 4 is used, no acetic acid is added) and 20 parts 3,7-dimethyl-3-octanol. Zeolites of Example 1 (0.24%) were added to the hydrogel blend. This mixture was sonicated until all components were dispersed (ca.30 minutes). The sonicated mixture was loaded to an eight cavity lens mold of the type described in U.S. Pat. No. 4,640,489 and cured for 1200 sec. Polymerization occurred under a nitrogen purge and was photoinitiated with visible light generated with a Philips TL 20W/03T fluorescent bulbs, at temperatures of 45 to 75° C. After curing, the molds were opened, and the lenses were released into 60% IPA, then leached in an IPA/DI water step down to remove any residual monomers and diluent. Finally, the lenses are equilibrated in either DI water or physiological borate-buffered saline to give Lenses A¹.

### EXAMPLE 3

### Preparation of Di-vinyl Ethylene Oxide Zeolites and Lenses B¹

Type A zeolites (10% silver 1000-2000 nm) were dried in a vacuum oven at 100° C. overnight and loaded into a modified plasma chamber as described by V. Panchalingam, X. Chen, C. R. Savage, R. B. Timmons and R. C. Eberhart, J. Appl. Polm. Sci.: Appl. Polym. Symp., 54, 123 (1994). This device was modified by replacing the stationary chamber with a rotating chamber. The dried zeolites were placed loaded into the rotating chamber and treated with an argon plasma pulsed at 10/100 milli-seconds on/off cycle (“ms cycle”) and 100 Watts for 15 minutes The argon treated zeolites were subsequently treated with EO₂V plasma, pulsed at 10/200 ms cycle and 100 W for 100 minutes. The resulting particles were removed from the chamber and passed through a 400-mesh stainless sieve. These filtered particles were treated a second time with EO₂V plasma, pulsed at 10/200 ms cycle and 100 W for 100 minutes and collected to give Zeolite B¹ as a solid. One percent (1.0%) of Zeolite B¹ was added to the hydrogel blend of Example 2. Once the zeolites were added, the mixture was treated and cured according to the method of Example 2 to give Lenses B¹.

### EXAMPLE 4

### Preparation of Uncoated Zeolites and Lenses C¹

Type A zeolite particles (15.0 grams, average particle size 1000 nm to 2000 nm) containing 10% silver by weight were added to the hydrogel blend of Example 2. Once the zeolites were added, the mixture was treated and cured according to the method of Example 2 to give Lenses C¹.

### Example 5

### Release Rates of Silver From Lenses A¹, B¹, and C¹.

Five (5) lenses were collected for silver analysis immediately prior to initiating the silver release study. Twenty-five (25) lenses were incubated individually in 20 ml polypropylene vials containing 2.2 ml of protein solution consisting of 1.8 mg/ml lysozyme, 1.8 mg/ml albumin, and 1.8 mg/ml gamma-globulins in saline solution. The vials were agitated on an orbital shaker at 100 r.p.m. Five lenses were recovered and pooled for analysis each day at approximately the same time of day. The remaining lenses were transferred to 2.2 ml of fresh protein solution. All samples and the five control lenses were dried in-vacuo at approximately 80° C. and analyzed for silver content by inductively coupled plasma atomic emission spectroscopy. The amount of silver content per lens was measured. The weight percentage of silver remaining in the lenses was calculated and is listed in Table 1.

TABLE 1

| | Lens C¹ | Lens B¹ | Lens A¹ |
|---|---|---|---|
| Day | silver content | silver content | silver content |
| 0 | 100% | 100% | 100% |
| 1 | 41% | 44% | 80% |
| 2 | 13% | 44% | 60% |
| 3 | 10% | 39% | 56% |
| 4 | <9% | 38% | 80% |
| 5 | <9% | 33% | 30% |

### EXAMPLE 6

### Release Rates of Silver From Lenses A², D¹, E¹ and F¹

Four different silanes were applied to the surface of type A zeolites particles having an average particle size of 1000 nm to 2000 nm and an initial silver content of 20%, using the method of example 1. The silanes are octadecyltrimethoxysilane, octyltrimethoxysilane, butyltrimethoxysilane, and acetoxypropyltrimethoxysilane, and they gave zeolites A², D¹, E¹ and F¹ respectively.

Approximately 0.05% of these zeolites were added to the hydrogel blend of Example 2 using the method of Example 2 to give Lenses A² D¹, E¹ and F¹ respectively The release assay of Example 5 was conducted and the data displayed in Table 2.

TABLE 2

| Time | Lens A² | Lens D¹ | Lens E¹ | Lens F¹ | Lens C¹ |
|---|---|---|---|---|---|
| Days | % Ag | % Ag | % Ag | % Ag | % Ag |
| 0 | 100 | 100 | 100 | 100 | 100 |
| 1 | 69.3 | 71.4 | 72.2 | 41.3 | 41 |
| 2 | 41.8 | 53.1 | 62.2 | 47.8 | 13 |
| 3 | 40.8 | 38.8 | 31.1 | 54.8 | 10 |
| 4 | 36.7 | 52.0 | 31.1 | 53.9 | <9 |
| 5 | 34.7 | 36.7 | 38.9 | 32 | <9 |

### EXAMPLE 7

### Preparation of Nanoscale Zeolites

Nanoscale zeolites were prepared with a tetramethylammonium template, using the procedure described by B. J. Schoeman et. al. In ZEOLITES, 1994, Vol. 14, February, 1994, p. 110-116, following the procedure to make A1, but without the addition of NaOH. Particle size analysis using a BECKMAN Coulter Particle Size Analyzer showed the particles to have a mean size of 164 nm with a standard deviation of 44 nm. These particles were rinsed three times with borate buffered saline solution, once with deionized water, three times with methanol, in each case isolating the zeolites by ultracentrifugation. 3.42 g of zeolite was suspended in 34.2 g methanol. 3.42 ml deionized water, 0.34 9 acetic acid, and 3.42 g octadecyltrimethoxysilane (OTS) were added. The suspension was stirred for 71 hours at room temperature, then rinsed three times with 25 ml methanol and ultracentrifuged. Silicone hydrogel lenses were made by combining 0.25% (wgt) of this OTS-treated nanozeolite with the hydrogel blend of Example 2 and lenses were prepared by the method of Example 2. These lenses were treated with silver by placing them into 5.0% aqueous silver nitrate solution at 45° C. for five minutes and subsequently rinsing them with DI.

### EXAMPLE 8

The procedure of Example 7 was followed, except using triethylamine amine in place of acetic acid to catalyze the OTS reaction.

### EXAMPLE 9

### Preparation of Lenses G¹

Using a procedure adapted from Chem. Mater. 5(6), 1993, 869-875, silver zeolite (2 g Type A zeolites, 20% Ag by weight), 200 mg of polybutadiene (average Mn=3,000, 0.066 mmole), and 20 mL of dichloromethane were charged in a 150 mL beaker flask. The apparatus was connected to a rotary evaporator and rotated for 30 minutes with the heating bath set at 40° C. The reaction mixture was cooled to room temperature and a solution of 60 mg of 2,21-azobisisobutyronitrile (0.375 mmole) in 5 mL of dichloromethane was added to the suspension. The flask was connected to the rotary evaporator, and the solvent was removed with rapid rotation, while maintaining the temperature below 20° C.

The solid reactant system was spread as a thin layer in a crystallizing dish. The vessel was covered with filter paper and placed in a vacuum oven at 100° C. for three hours to crosslink the polybutadiene coating. The yield was 1.85 g of white hydrophobic material (84.09%, Note—the zeolite used in the process had a water content of around 10% by weight, isolated yield is greater than that reported (closer to 92%)).

The coated zeolite (0.5% w/w) was dispersed into the hydrogel blend of Example 2 and lenses were fabricated using the method of Example 2 to give lens G¹. The release assay of Example 5 was carried out and the results are tabulated in Table 3.

### EXAMPLE 10

### Preparation of Lenses H¹

2 grams of zeolite containing 10% Ag zeolite (Type A having an average particle-diameter of 1000 to 2000 nm), 50 mL methylene chloride, 500 mL H₂O, 100 mL triethylamine were combined in a 250 mL beaker and stirred until uniform consistency achieved (typically 30-60 min). 250 mL of octadecyltrichlorosilane was added every 15 minutes to a total of 2 mL of silane (8 additions-2 hours). The sample was filtered using the following procedure: 1) vacuum filter to dry powder, 2) re-suspend in methylene chloride, shaking vigorously, 3) repeat (1) and (2) 4 times. After the fourth filtration procedure the isolated solid dried under vacuum for 4 hr at room temperature. Prior to use, the zeolite powder was ground with a mortar and pestle.

The coated silane was added to the hydrogel blend of Example 2 and lenses were formed using the method of Example 2 to give lens H¹. The release assay of Example 5 was carried out and the results are tabulated in Table 3.

TABLE 3

| Time | Lens G¹ | Lens H¹ | Lens C¹ |
|---|---|---|---|
| Days | Ag % | Ag % | Ag % |
| 0 | 100 | 100 | 100 |
| 1 | 48.9 | 39.4 | 41 |
| 2 | 29.3 | 32.9 | 13 |
| 3 | 30.1 | 28.8 | 10 |
| 4 | 31.8 | 31.8 | <9 |
| 5 | 27.9 | | <9 |

### EXAMPLE 11

### Biological Vortex Assay Results

Lenses were made from the hydrogel blend of Example 2 with 0.5% OTS-treated zeolite containing 20% silver. The lenses were tested using the biological vortex assay described above. The number of viable bacteria found in the assay was reduced by 99.7%.

### EXAMPLE 12

### Alternative Monomer Formulations

Rase Monomer Formations

Formulations B—R, listed in Table 4, are the base monomer mixes (all amounts are calculated as weight percent of the total weight of the monomer mix combination). The coated zeolites (0.0005% w/w (50 ppm) to about 1.0% w/w) of the invention may be added to all of the compositions of Table 1 and contact lenses may be prepared according to the following method.

Contact Lens Formation

The blends are sonicated at 25-37° C. until all components are dissolved or dispersed (30-120 minutes) and are subsequenty loaded to an eight cavity lens mold of the type described in U.S. Pat. No. 4,640,489 and cured for 1200 sec.

TABLE 4

| | Formulation | | | | | | | | | | | | | | | |
|---|---|---|---|---|---|---|---|---|---|---|---|---|---|---|---|---|
| | B | C | D | E | F | G | H | I | J | K | M | N | O | P | Q | R |
| | Macromer | | | | | | | | | | | | | | | |
| | 2 | 3 | 3 | 2 | 2 | 1 | 2 | 2 | 2 | 2 | 2 | 2 | 2 | 2 | 2 | 2 |
| [Macromer] | 25.00 | 60.00 | 20.00 | 17.98 | 17.98 | 30.00 | 19.98 | 17.98 | 17.98 | 19.98 | 40.00 | 18.00 | 18.00 | 18.00 | | |
| TRIS | 18.00 | 0.00 | 40.00 | 21.00 | 21.00 | 0.00 | 8.00 | 20.00 | 25.00 | 20.00 | 20.00 | 14.00 | 14.00 | 14.00 | | |
| DMA | 28.00 | 36.00 | 36.00 | 25.50 | 25.50 | 27.00 | 26.00 | 22.00 | 9.00 | 23.00 | 35.00 | 26.00 | 26.00 | 26.00 | | |
| mPDMS | 18.00 | 0.00 | 0.00 | 21.00 | 21.00 | 39.00 | 28.50 | 25.50 | 30.00 | 28.50 | | 28.00 | 28.00 | 28.00 | | |
| Norbloc | 2.00 | 3.00 | 3.00 | 2.00 | 2.00 | 2.00 | 2.00 | 2.00 | 2.00 | 2.00 | 3.00 | 2.00 | 2.00 | 2.00 | | |
| CGI 1850 | 1.00 | 1.00 | 1.00 | 1.00 | 1.00 | 2.00 | 1.00 | 1.00 | 1.00 | 1.00 | 2.00 | 1.00 | 1.00 | 1.00 | | |
| TEGDMA | 0.00 | 0.00 | 0.00 | 1.50 | 1.50 | 0.00 | 1.50 | 1.50 | 0.50 | 1.50 | | | 0.25 | 0.50 | | |
| HEMA | 0.00 | 0.00 | 0.00 | 5.00 | 5.00 | 0.00 | 5.00 | 5.00 | 7.00 | 5.00 | | 5.00 | 5.00 | 5.00 | 96.8 | 98.6 |
| Blue HEMA | 0.00 | 0.00 | 0.00 | 0.02 | 0.02 | 0.00 | 0.02 | 0.02 | 0.02 | 0.02 | | | | | 0.02 | |
| PVP | 8.00 | 0.00 | 0.00 | 5.00 | 5.00 | 0.00 | 8.00 | 5.00 | 7.50 | 9.00 | | 5.00 | | | | |
| Darocur | | | | | | | | | | | | | | | 0.3 | 0.30 |
| 1173 | | | | | | | | | | | | | | | | |
| EGDMA | | | | | | | | | | | | | | | 0.8 | 0.8 |
| TMPTMA | | | | | | | | | | | | | | | 0.1 | 0.1 |
| MAA | | | | | | | | | | | | | | | 2.0 | |
| Diluent % | 20 | 20 | None | 20 | 50.00 | 41.00 | 37.50 | 20.00 | 40.00 | 50.00 | 20.00 | 20.00 | 20.00 | 20.00 | 52.00 | 52.00 |
| Diluent | 3M3P | 3M3P | NA | D30 | TAA | 3M3P | 3M3P | TAA | 3M3P | 3M3P | D30 | D30 | D30 | D30 | BAGE | BAGE |

### EXAMPLE 13

### Preparation of Lenses Containing an Oxidizing Agent

A hydrogel blend was made from the following monomer mix (all amounts were calculated as weight percent of the total weight of the combination): 17.98% Macromer 2, 28.0% mPDMS, 14.0% TRIS, 26.0% DMA, 5.0% HEMA, 1.0% TEGDMA, 5.0% PVP, 1.0% CGI 1850, and 2.0% Norbloc, blended with D3O as a diluent in a ratio of 80 parts mixture with 20 parts diluent. To this blend was added 1.0 part acetic acid, 1000 ppm (wt) A-type zeolite containing 20% (wt) silver and 354 ppm hydrogen peroxide. This mixture was sonicated until ail components were dispersed (ca.45 minutes). The sonicated mixture was loaded to an eight cavity lens thermoplastic mold and cured for 1200 sec. Polymerization occurred under a nitrogen purge and was photoinitiated with visible light generated with a Philips TL 20W/03T fluorescent bulbs, curing for 25 minutes at 50° C. After curing, the molds were opened, and the lenses were released into 50% IPA in water, then leached in IPA to remove any residual monomers and diluent. Finally, the lenses are equilibrated in physiological borate-buffered saline. After four days at room temperature these lenses were colorless, as compared to lenses that were made without addition of H₂O₂, which had developed a visible brown color. Additional concentrations of hydrogen peroxide tested as described above and the observations of lens color are listed in Table 5.

TABLE 1
Hydrogen peroxide added to monomer mix.

| Example | ppm added H₂O₂ | lens appearance |
|---|---|---|
| 1 | 354 | colorless |
| 2 | 177 | colorless |
| 3 | 105 | colorless |

### EXAMPLE 14

### Treatment of Lenses with an Oxidizing Agent

Lenses were made following example 13, but with addition of 0.25% (wt) type-A zeolite containing 20% (wt) silver and without addition of hydrogen peroxide to the monomer mix. These lenses were placed into optically transparent cells containing test or control lens storage solution. The lenses were then either stored under a bank of fluorescent lights-for two months. The test solution was a solution of sodium borate, boric acid, and sodium perborate sufficient to generate up to 0.006% hydrogen peroxide (sold under the trade name Quick Care FINISHING SOLUTION by CIBA Vision Corporation), and the test solution was borate buffered saline without the sodium perborate. The color of the lenses was measured using the CIELAB convention with an portable sphere spectrophotometer from X-Rite, Incorporated. The L, a, and b values of three lens measurements were averaged and are reported in Table 6. The small changes in the a and b values of the perborate-treated stored lenses, as compared to the saline-stored lenses, illustrate that the perborate prevents discoloration of the lenses. The b color coordinate indicates the amount of yellowness (higher positive b value=more yellowness) in a given, material or its blueness (lower negative b value=more blueness). Comparison of the b values of Table 6 show that the yellowing of lenses is prevented in perborate containing solution.

TABLE 6
L, a, and b values for light-exposed lenses

| Storage solution | L value | a value | b value |
|---|---|---|---|
| before aging | 84.5 ± 1.3 | −0.57 ± 0.4 | 7.98 ± 2.3 |
| saline | 84.8 ± 0.7 | −4.06 ± 0.6 | 20.0 ± 3.9 |
| perborate | 85.6 ± 0.6 | −1.12 ± 0.4 | 8.45 ± 1.9 |

### EXAMPLE 15

### Treatment of Lenses with an Oxidizing Agent

Lenses were prepared as in Example 13, but without addition of silver-zeolite or addition of hydrogen peroxide to the monomer mix. These lenses were placed into commercial foil-sealed polypropylene lens packages, along with 10 μl of of a 0.10% aqueous solution of H₂O₂, 20 μl of a solution of Age (0.75 wt % Ag), and diluted with a solution of 9.26 g/L boric acid, 1.86 g/L sodium borate and an appropriate surfactant in water to a total volume of 1.0 ml. The sealed lenses were autoclaved for 30 minutes at 121° C. The solution was colorless, as compared to a comparative experiment which omitted the H₂O₂, in which the solution was visibly yellow.

### EXAMPLE 16

### Treatment of Lenses with an Oxidizing Agent

Lenses were made as in Example 13, but with 1000 ppm of 10% (wt) silver-zeolites, and without the hydrogen peroxide in the monomer mix. The lenses were placed individually into glass vials with 2 ml borate-buffered saline containing 1.5% H₂O₂. The lenses were observed over a period of 48 hours, over which time they remained colorless. Analysis for silver immediately after lens formation and 48 hours later showed no drop in silver level. The lenses exhibited a 1.7 log drop in viable bacteria in the vortex assay described above as compared to lenses without the silver-zeolites and not treated in H₂O₂.

### EXAMPLE 17

### Dispersion of Monomer Formulations with Particulate Matter

A dispersion that may be used to form some of the lenses of the invention, such as lenses containing silver salts, Momomer of 030 that are bound to silver, or zeolites, is prepared by the following method. Once formed this dispersion may be cured using the methods of Example 1.

I. Pre-Dispersion

1. Sterilize mixing vessel and cover.
2. Pre-mix the dry silver complex in the liquid formulations at slow speed ensuring minimal heat build up. Keep container covered to preclude light and contamination.
3. Slowly increase speed to breakdown agglomerates (Note: Do not allow heat to build up.).
   II. Dispersion
1. Thoroughly clean mill with isopropyl alcohol. Allow to air dry. Assist with heat if necessary.
2. Hook up stainless steel intake and outlet lines from mixing vessel to mill and from mill to empty, sterilized, covered vessel.
3. Load sterilized media into mill.
4. Process material through horizontal temperature controlled media mill.
5. Speed of mill and speed of media and temperature of material to be adjusted to achieve desired dispersion.
6. Steps #4 and #5 to be repeated until material achieves the required finished dispersion. Dispersion to be determined by microscopic evaluation.

### EXAMPLE 18

### Movement of Lenses

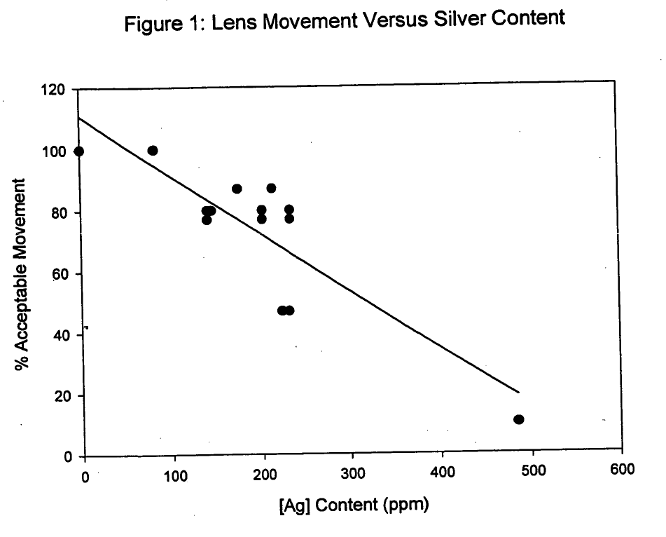

Lenses were prepared using the method of Example 2. All lenses contained 0.25 weight percent of Type A zeolites. The zeolites of entries 2-13 contain 20% active silver by weight based upon the weight of the added zeolites. The silver content of entry 1 was 10% active silver by weight based upon the weight of the added zeolites. In addition, the zeolites of entry 1 were coated with EO₂ V as described in Example 3. Entry 14 was prepared using 0.25% of a Type A zeolite that contains sodium instead of silver. This zeolite was coated with OTS using the method of Example 1 and subsequently treated with a silver solution before it was incorporated into the lens formulation of example 2. Prior to insertion in patient's eyes, the amount of silver in the lenses was determined by inductively coupled plasma atomic emission. The movement of each lens type was tested on ten (10) subjects per type of lens using the push up assay (Contact Lens Practice, Chapman & Hall, 1994, edited by M. Ruben and M. Guillon, pgs. 589-99). All lenses were evaluated 30 minutes after placing the lenses on patients' eyes. The percentage of lenses having acceptable movement qualities was calculated as follows. Any lens having a score of greater than −2 on the push up test was an acceptable lens. In a each patient study, the number of acceptable lenses was divided by the total number lenses. Lenses having a percentage of movement equal to or greater than 50% are acceptable. In addition, prior to insertions in a patient's eyes the efficacy of the lenses tested using the Vortex Assay. The activity of the lenses in these assays is listed in Table 7 as the log reduction of the assay. FIG. 1 shows the percentage lenses having acceptable movement vs the amount of silver in each lens.

TABLE 7

| Entry | [Ag ppm] | Log Reduction |
|---|---|---|
| 1 | 83 | N/A |
| 2 | 141 | N/A |
| 3 | 202 | N/A |
| 4 | 234 | 1.6 |
| 5 | 141 | 0.9 |
| 6 | 146 | N/A |
| 7 | 145 | N/A |
| 8 | 202 | N/A |
| 9 | 234 | 1.6 |
| 10 | 232 | 1.4 |
| 11 | 224 | 1.3 |
| 12 | 175 | 0.7 |
| 13 | 214 | 0.7 |
| 14 | 485 | 2.5 |

N/A not available

1. An antimicrobial lens comprising a zeolite coated with at least one hydrophobic substance that slows the release of the antimicrobial metal from the zeolite.

2. The antimicrobial lens of claim 1, wherein the zeolite is coated with a composition comprising at least one silane.

3. The lens of claim 1 wherein the zeolite comprises silver.

4. The lens of claim 1 wherein the lens is a contact lens.

5-17. (canceled)

18. The lens of claim 1 having
more than about 0.02 weight percent zeolite, and
less about 1.0 weight percent zeolite.

19. The lens of claim 1 having
more than about 0.025 weight percent zeolite, and
less about 0.1 weight percent zeolite.

20. The lens of claim 1 having
more than about zero weight percent zeolite, and
less than about 0.1 weight percent zeolite.

21.-22. (canceled)

23. The lens of claim 2 wherein the coated zeolites comprise at least two different hydrophobic substance.

24. (canceled)

25. (canceled)

26. The antimicrobial lens of claim 1 wherein the hydrophobic compound comprises at least one hydrophobic monomer.

27. The lens of claim 26 wherein the hydrophobic monomer is selected from the group consisting of-perfluoropropylene oxide, diethylene glycol vinyl ether, methyl methacrylate, lauryl methacrylate, styrene, 1,3-butadiene, propylene glycol, hexamethylcyclotrisiloxane, and mixtures thereof.

28. The lens of claim 26 wherein the hydrophobic monomer is selected from the group consisting of perfluoropropylene oxide, diethylene glycol vinyl ether and mixtures thereof.

29. The lens of claim 26 having
more than about 0.02 weight percent zeolite, and
less about 1.0 weight percent zeolite.

30. The lens of claim 26 having
more than about 0.025 weight percent zeolite, and
less about 0.1 weight percent zeolite.

31. The lens of claim 26 having
more than about zero weight percent zeolite, and
less than about 0.1 weight percent zeolite.

32.-52. (canceled)
